(19) United States
(12) Patent Application Publication
(10) Pub. No.: US 2010/0249249 A1
(43) Pub. Date: Sep. 30, 2010

(54) Reduction of the Formation of Biofilm by Means of Multifunctional Copolymers

(76) Inventors: Stefan Stumpe, Dusseldorf (DE); Mirko Weide, Dusseldorf (DE); Roland Breves, Mettmann (DE); Noelle Wrubbel, Dusseldorf (DE); Matthias Lüken, Dusseldorf (DE); Julia Boy, Mulheim an der Ruhr (DE)

Correspondence Address: Henkel Corporation, 10 Finderne Avenue, Suite B, Bridgewater, NJ, 08807, US

(21) Appl. No.: 12/792,876

(22) Filed: Jun. 3, 2010

Related U.S. Application Data
(63) Continuation of application No. PCT/EP08/65972, filed on Nov. 21, 2008.

(30) Foreign Application Priority Data
Dec. 3, 2007 (DE) 10 2007 058 342.9

(51) Int. Cl. C09D 5/00; C11D 3/37; A61K 47/32; A61K 8/81

(52) U.S. Cl. 514/772.4; 524/555; 524/547; 526/279; 510/475

(57) ABSTRACT

Multifunctional copolymers, washing and cleaning agents comprising said copolymers, and use of those copolymers for reduction of the attachment of microorganisms and/or for reduction of the formation of biofilm on surfaces.

### CROSS REFERENCE TO RELATED APPLICATIONS

The present application is a continuation of International Application No. PCT/EP2008/065972 filed 21 Nov. 2008, which claims priority to German Patent Application No. 10 2007 058 342.9 filed 3 Dec. 2007.

The present invention relates to multifunctional copolymers, washing and cleaning agents comprising said copolymers, and methods of using those copolymers to reduce the attachment of microorganisms and/or reduce the formation of biofilm on surfaces.

There is a need in a variety of areas for agents that prevent adhesion of microorganisms and/or development of biofilms.

In households, for example, mold can be found in many different places such as in the kitchen or in moist areas such as the bathroom. Molds give rise to significant problems because spores released by them into the atmosphere are often the cause of allergies. Moreover, bacteria can develop strong smelling and unaesthetic biofilms on surfaces in a household, especially in pipe work. Extensive biofilm formation can block the pipes and other flow systems. Combating fungi and bacteria with biocides involves an increased risk of biocidal resistance, so that after some time new antimicrobials have to be found which are effective against these resistant microorganisms. Furthermore, biocides are not always ecologically and toxicologically harmless, and can be inadequate for attacking a well-developed biofilm.

Moreover, delicate textiles such as silk or microfibers are being used more and more frequently for clothing that can only be washed at 30 or 40° C. Consequently, fungi such as the human pathogen Candida albicans and bacteria are not killed off. Particularly after a fungal infection, fungi that have not been destroyed and adhere to clothing can cause a re-infection.

In addition, denture wearers often contract an oral candidosis (moniliosis). Fungus cells that adhere to the surface of the prosthesis can, through contact, colonize the mucous membranes often already damaged by pressure marks.

Up to now, antimicrobials that either inhibit the growth of microorganisms (bacteriostatic agents) or kill them off (biocides) have been employed to prevent any re-infection from microorganisms that adhere to clothing or to plastic surfaces. This is disadvantageous; as such biocides or bacteriostatic agents employed, for example, in washing and cleaning agents pollute the waste water, thereby impairing the operation of the microbial purification steps in waste water treatment plants. In addition, the selection pressure on microorganisms strongly increases their resistance such that after some time new antimicrobials have to be found which are effective against these resistant microorganisms. Accordingly, instead of biocides or bacteriostatically active substances, it is desirable to have biorepulsive substances available that prevent any adhesion but that do not physiologically impair the microorganisms.

Moreover, the reduction in adhesion by reducing contact of the human body with the microorganisms (e.g., in the respiratory system with mold spores) can also reduce the allergy triggering potential.

Another important field of application in which adhesion of microorganisms plays a decisive role is submersed surfaces in marine environments. Over time, sessile organisms colonize these surfaces in a generally defined sequence. Bacteria, fungi, microalgae and protozoa initially form a biofilm onto which larger organisms such as algae can form colonies. If the colonized surfaces concern those of industrial equipment or ships, then obviously protective measures should be taken as uneven surfaces due to the colonization increases friction resistance and thereby fuel consumption; moreover, colonized material corrodes more easily. The organotin-containing antifouling paints previously used are very efficient but are also highly toxic and non-specific. Their application was forbidden in 2003 in the “International Convention on the control of harmful Antifouling Systems”, and since 2008 there is a ban on usage. This led to an increased interest in development of more environmentally compatible antifouling methods.

Accordingly, the present invention provides a method for reducing the adhesion of microorganisms on surfaces and/or inhibiting the development of biofilms without loading these surfaces or the waste water with biocidal and/or bacteriostatic active substances.

It has now been surprisingly found that adhesion of microorganisms on surfaces, particularly the formation of biofilm, can be reduced in a simple manner with the use of certain copolymers of functional monomers containing unsaturated groups. This can be achieved, for example, by incorporating these copolymers in a cleaning agent or treatment agent used to treat the surface in question. Alternatively, the copolymers can be introduced and/or incorporated into the material whose surface is intended to be protected from the adhesion.

### BRIEF DESCRIPTION OF THE DRAWINGS

FIG. 1 is a graph illustrating the results of an adhesion test described in Example 3 below.

FIG. 2 is a graph illustrating the results of an adhesion test described in Example 4 below.

FIG. 3 is a graph illustrating the results of an adhesion test described in Example 5 below.

The copolymers are available by copolymerizing the following ethylenically unsaturated monomers in the given amounts:
5-95 wt. % of at least one ethylenically unsaturated compound containing an anionic group (hereinafter also called “vinyl monomer (A)” or “anionic vinyl monomer (A)”;
0-50 wt. % of at least one ethylenically unsaturated compound containing a secondary or tertiary amino group or a quaternary ammonium group (hereinafter also called “vinyl monomer (B)” or “vinyl monomer (B) containing a secondary or tertiary amino group or a quaternary ammonium group”);
5-95 wt. % of at least one non-ionic hydrophilic ethylenically unsaturated compound (hereinafter also called “vinyl monomer (C)” or “non-ionic hydrophilic vinyl monomer (C)”);
0-15 wt. % of at least one polyfunctional hydrophilic ethylenically unsaturated compound (hereinafter also called “vinyl monomer (F)” or “polyfunctional hydrophilic vinyl monomer (F)”);
0-30 wt. % of at least one hydrophobic ethylenically unsaturated compound (hereinafter also called “vinyl monomer (D)” or “hydrophobic vinyl monomer (D)”); and
0-20 wt. % of at least one ethylenically unsaturated compound containing at least one silicone group (hereinafter also called “vinyl monomer (E)” or “vinyl monomer (E) comprising at least one silicone group”);
   wherein the sum of monomers (A), (B), (C), (D), (E) and (F) is 100 wt. %.

In a preferred embodiment, the quantities are selected as follows:
65-95 wt. % of at least one anionic vinyl monomer (A);
0-5 wt. %, preferably 0-2 wt. %, of at least one vinyl monomer (B) containing a secondary or tertiary amino group or a quaternary ammonium group;
5 to 25 wt. % of at least one non-ionic hydrophilic vinyl monomer (C);
0 to 2 wt. % of at least one polyfunctional vinyl monomer (F);
0-2 wt. % of at least one hydrophobic vinyl monomer (D); and
0-2 wt. % of at least one vinyl monomer (E) comprising at least one silicone group;
   wherein the sum of monomers (A) and (C) is 80 wt. % or greater, preferably 90 wt. % or greater, and the sum of monomers (A), (B), (C), (D), (E) and (F) is 100 wt. %; wherein in a preferred embodiment, the sum of monomers (A) and (C) is at least 98 or 99 wt. %, preferably 100 wt. %.

In another preferred embodiment, the quantities are selected as follows:
5-30 wt. %, preferably 15-25 wt. %, of at least one anionic vinyl monomer (A);
0-5 wt. %, preferably 0-2 wt. %, of at least one vinyl monomer (B) containing a secondary or tertiary amino group or a quaternary ammonium group;
70 to 95 wt. %, preferably 75-85 wt. %, of at least one non-ionic hydrophilic vinyl monomer (C);
0-2 wt. % of at least one polyfunctional vinyl monomer (F);
0-2 wt. % of at least one hydrophobic vinyl monomer (D); and
0-2 wt. % of at least one vinyl monomer (E) comprising at least one silicone group;
   wherein the sum of monomers (A) and (C) is 80 wt. % or greater, preferably 90 wt % or greater, and the sum of monomers (A), (B), (C), (D), (E) and (F) is 100 wt. %; wherein in a preferred embodiment, the sum of monomers (A) and (C) is at least 98 or 99 wt. %, preferably 100 wt. %.

In a quite particularly preferred embodiment, the copolymer is a copolymer obtained by copolymerization of—
5-30 wt. %, preferably 15-25 wt. %, particularly preferably 18-22 wt. %, of at least one anionic vinyl monomer (A); and
70-95 wt. %, preferably 75-85 wt. %, particularly preferably 78-82 wt. %, of at least one non-ionic hydrophilic vinyl monomer (C);
   wherein the sum of monomers (A) and (C) is at least 99 wt. %, preferably 100 wt. %.

The present invention therefore provides a method for temporarily or permanently reducing the adhesion of microorganisms on surfaces and/or reducing formation of a biofilm on surfaces, wherein a copolymer according to the invention is applied onto the surface or is incorporated into the materials, whose surfaces are intended to be protected from adhesion.

The present invention therefore also provides a copolymer according to the invention for (temporarily or permanently) reducing the adhesion of microorganisms on surfaces and/or reducing formation of a biofilm on surfaces.

“Reducing the attachment or adhesion” is understood to mean a significant reduction of the number of attached microorganisms. Thus, the number of attached microorganisms is preferably reduced by 20 or 40% or greater, particularly preferably by 50, 60, 70 or 80% or greater, in particular by 90 or 95% or greater with respect to an untreated control sample. Ideally, the adhesion is completely or almost completely prevented. Percentages refer to the difference in total mass of the adhered microorganisms based on a comparison of untreated and inventively treated surfaces.

### Inventive Copolymers—

The inventive copolymers and their components are described in more detail below.

Copolymers according to the present invention can be obtained by all polymerization processes for ethylenically unsaturated monomers known to one skilled in the art. The polymerization process is preferably carried out in the presence of thermo labile initiators, redox initiators or photo initiators at a temperature from 30° C. to 110° C. A hydrophilic solvent (e.g., water or a mixture of water with an additional hydrophilic solvent) is preferably used as the reaction medium. The reaction is preferably carried out in an atmosphere of inert gas such as nitrogen.

Average molecular weight of the inventive copolymers is preferably from 10,000 to 1,000,000 g/mol, particularly preferably from 40,000 to 300,000 g/mol. The inventive copolymers are preferably capable of furnishing hard surfaces with a hydrophilic, preferably negatively charged, coating. Moreover, the inventive copolymers are preferably capable of conferring a glossy appearance to ceramic surfaces. Preferred inventive copolymers on application result in surfaces with a surface energy of 50 mN/m or greater, preferably 75 mN/m or greater, and exhibit contact angles (with water) of preferably 30° or less, especially 10° or less, and contact angles (with diiodomethane) of preferably 40° or less, especially 20° or less.

### Monomers—

Inventively preferred embodiments of monomers (A), (B), (C), (D), (E) and (F) are exemplified below.

### Monomer (A)—

Inventively usable anionic vinyl monomers (A) include ethylenically unsaturated monomers having at least one anionic group or having at least one group that is negatively charged due to salt formation. Examples include monomers containing carboxylic groups and their salts, as well as monomers containing sulfonic acid groups and their salts.

Salts of the described monomers are preferably an alkali metal salt or an ammonium salt. In particular, they include sodium salts, potassium salts, ammonium salts, ethanolammonium salts and trimethylammonium salts. If salts are used, they can be used alone or in combination with free acids.

Furthermore, salts of the copolymers can be obtained, for example, by neutralization of the acid groups carried by the copolymers with alkali metal hydroxide or ammonium hydroxide.

Monomers containing carboxyl groups and their salts include acrylic acid, methacrylic acid, maleic acid, fumaric acid, sodium acrylate, potassium acrylate, sodium methacrylate, potassium methacrylate, sodium maleate, potassium maleate, sodium fumarate, potassium fumarate, ammonium acrylate, ammonium methacrylate, ammonium maleate, ammonium fumarate, acrylic acid monoethanolammonium salt, methacrylic acid monoethanolammonium salt, maleic acid monoethanolammonium salt and fumaric acid monoethanolammonium salt. Acrylic acid, methacrylic acid, sodium acrylate, sodium methacrylate, the monoethanolammonium salt of acrylic acid and the monoethanolammonium salt of methacrylic acid are preferred.

In particular, monomers having a structure corresponding to general Formulae (I) or (II) as well as their alkali metal and ammonium salts can be employed as sulfonic acid group-containing monomers.

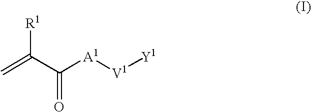

In general Formula (I), R¹ is hydrogen, methyl or ethyl, Y¹ is a sulfonic acid group (—SO₃H) or sulfonate group, A¹ is O or NH, and V¹ is a linear or branched, saturated or unsaturated hydrocarbon group containing 1 to 15 carbon atoms.

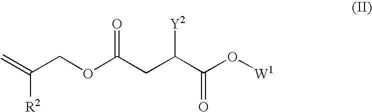

In general Formula (II), R² is hydrogen, methyl or ethyl, Y² is a sulfonic acid group (—SO₃H) or sulfonate group, and W¹ is a linear, branched or alicyclic, saturated or unsaturated hydrocarbon group containing 1 to 20 carbon atoms.

Each of the cited monomers can be used singly or in mixtures.

### Monomers (B)—

In a preferred embodiment, vinyl monomers containing secondary or tertiary amino groups or quaternary ammonium groups (B) correspond to compounds according to general Formula (III).

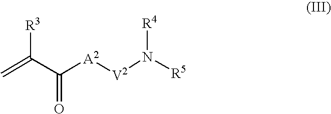

In general Formula (III), R³ is hydrogen, methyl or ethyl, A² is O or NH, and V² is a linear or branched, saturated or unsaturated hydrocarbon group containing 1 to 15 carbon atoms. R⁴ is hydrogen, methyl, ethyl, propyl or butyl and R⁵ is methyl, ethyl, propyl or butyl.

In particular, 2-tert-butylaminoethyl acrylate, 2-tert-butylaminoethyl methacrylate, dimethylaminoethyl acrylate, dimethylaminoethyl methacrylate (DMEMA), dimethylaminopropyl acrylate, dimethylaminopropyl methacrylate, dimethylaminobutyl acrylate, dimethylaminobutyl methacrylate, diethylaminoethyl acrylate, diethylaminoethyl methacrylate, dimethylaminoethylacrylamide, dimethylaminoethylmethacrylamide, dimethylaminopropylacrylamide (DMAPA), dimethylaminopropylmethacrylamide (DMAPMA), dimethylaminobutylacrylamide, dimethylaminobutylmethacrylamide, diethylaminoethylacrylamide or diethylaminoethylmethacrylamide can be used as vinyl monomers according to general Formula (III). DMAPA or DMAPMA are preferably used, with DMAPMA particularly preferably used.

Other preferred compounds include dimethylaminoethyl methacrylate, dimethylaminoethyl acrylate, dimethylaminoethylmethacrylamide and dimethylaminoethylacrylamide, wherein dimethylaminoethyl methacrylate (DM) and dimethylaminoethylmethacrylamide are particularly preferred.

Preferably, compounds according to general Formula (IV) or (V) are employed as the monomers containing quaternary ammonium groups.

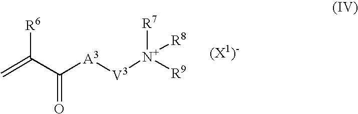

In general Formula (IV) R⁶ is hydrogen, methyl or ethyl, A³ is O or NH, V³ is a linear or branched, saturated or unsaturated hydrocarbon group containing 1 to 15 carbon atoms, R⁷, R⁸ and R⁹ are each independently methyl or ethyl, and X¹ is a counter ion.

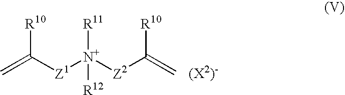

In general Formula (V) the R¹⁰ groups each independently are hydrogen, methyl or ethyl, R¹¹ and R¹² are independently methyl or ethyl, and X² is a counter ion. Z¹ and Z² are independently methylene, ethylene or propylene.

The counter ion in Formulae (IV) and (V) can be, for example, a halide, sulfate or anion of an organic acid, with chloride, bromide, sulfate and citrate being particularly preferred counter ions.

Exemplary inventively employable compounds corresponding to Formula (IV) include: acryloxyethyltrimethyl-ammonium chloride, methacryloxyethyltrimethyl-ammonium chloride, acryloxypropyltrimethyl-ammonium chloride, methacryloxypropyltrimethyl-ammonium chloride, acryloxybutyltrimethyl-ammonium chloride, methacryloxybutyltrimethyl-ammonium chloride, acryloxyethyltriethyl-ammonium chloride, methacryloxyethyltriethyl-ammonium chloride, acrylamidoethyltrimethyl-ammonium chloride, methacrylamidoethyltrimethyl-ammonium chloride, acrylamidopropyltrimethyl-ammonium chloride, methacrylamidopropyltrimethyl-ammonium chloride, acrylamidobutyltrimethyl-ammonium chloride, methacrylamidobutyltrimethyl-ammonium chloride, acrylamidoethyltriethyl-ammonium chloride and methacrylamidoethyltriethyl-ammonium chloride. Acryloxyethyltrimethyl-ammonium chloride, methacryloxyethyltrimethyl-ammonium chloride, acrylamidopropyltrimethyl-ammonium chloride (AAPTAC) and/or methacrylamidopropyltrimethyl-ammonium chloride (MAPTAC) are preferably used, with acryloxyethyltrimethyl-ammonium chloride, MAPTAC and/or AAPTAC being particularly preferably used.

Exemplary inventively usable compounds corresponding to Formula (V) include diallyldimethyl-ammonium chloride (DADMAC), diallyldimethyl-ammonium bromide, diallyldiethyl-ammonium chloride and diallyldiethyl-ammonium bromide. Diallyldimethyl-ammonium chloride, diallyldimethyl-ammonium bromide are preferably employed, with diallyldiethyl-ammonium bromide being particularly preferably employed.

According to the invention, single compounds from vinyl monomers containing tertiary amino groups or quaternary ammonium groups as well as any combinations thereof can be employed.

### Monomers (C)—

Hydrophilic vinyl monomers (C) are preferably compounds according to general Formula (VI)—

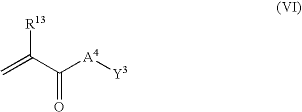

wherein R¹³ is hydrogen, methyl or ethyl; A⁴ is O or NH; Y³ preferably is (CH₂CH₂O)ₙ₁B¹, wherein n¹ preferably is a number from 1 to 120, in particular from 1 to 60, and B¹ preferably is hydrogen or methyl. Instead of polyethyleneoxy groups, the inventive vinyl monomers can also possess other polyalkyleneoxy groups, especially copolymers of polyethyleneoxy and polypropyleneoxy and/or polybutyleneoxy groups

Methoxypolyethylene glycol methacrylate (with n¹=1 to 30), and preferably methoxypolyethylene glycol methacrylate (with n¹=4, 7, 9, 11, 17, 22, 23 or 45), may be cited as examples of compounds of the general Formula (VI).

The molecular weight of monomers (C) is preferably up to 15,000 g/mol, particularly preferably from 300 to 12,000 g/mol, and above all 300 to 2500 g/mol.

### Monomers (D)—

Hydrophobic vinyl monomers (D) possess hydrophobic properties and preferably have a structure corresponding to general Formula (VII)—

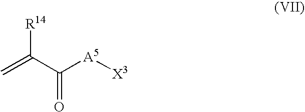

wherein R¹⁴ has the same meaning as R¹³ in general Formula (VI), A⁵ has the same meaning as A⁴ in general Formula (VI) and X³ is a linear or branched, saturated or unsaturated hydrocarbon group containing 1 to 15 carbon atoms.

Exemplary inventively employable compounds corresponding to Formula (VII) include: alkyl (meth)acrylates and alkyl(meth)acrylamides, especially methyl acrylate, methyl methacrylate (MMA), ethyl acrylate, ethyl methacrylate, propyl acrylate, propyl methacrylate, butyl acrylate, butyl methacrylate, t-butyl acrylate, t-butyl methacrylate, hexyl acrylate, hexyl methacrylate, 2-ethylhexyl acrylate, 2-ethylhexyl methacrylate (2EHMA), octyl acrylate, octyl methacrylate, lauryl acrylate, lauryl methacrylate, propylacrylamide, propylmethacrylamide, butylacrylamide, butylmethacrylamide, hexylacrylamide, hexylmethacrylamide, octylacrylamide, octylmethacrylamide, laurylacrylamide and laurylmethacrylamide. Propyl acrylate, propyl methacrylate, butyl acrylate, butyl methacrylate, t-butyl acrylate, t-butyl methacrylate, hexyl acrylate, hexyl methacrylate, 2-ethylhexyl acrylate and 2-ethylhexyl methacrylate are inventively preferred, with butyl acrylate, butyl methacrylate, t-butyl acrylate and t-butyl methacrylate being particularly preferred.

Single hydrophobic vinyl monomers (D) as well as their mixtures can be used.

Vinyl monomers (E) containing at least one silicone group are preferably monomers according to general Formula (VIII)—

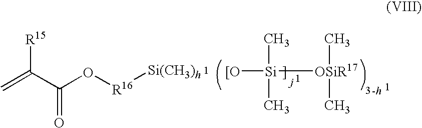

wherein R¹⁵ is hydrogen, methyl or ethyl, R¹⁶ is a linear or branched, saturated or unsaturated hydrocarbon group containing 1 to 6 carbon atoms, preferably methylene, ethylene, propylene or butylene, wherein one or more CH₂ groups in the hydrocarbon group can also optionally be substituted by O, R¹⁷ is a linear or branched, saturated or unsaturated hydrocarbon group, preferably a linear, saturated hydrocarbon group containing 1 to 30, preferably 1 to 22 carbon atoms, wherein the hydrocarbon group can optionally also be mono- or polysubstituted by fluorine, and wherein h¹ is 1 or 2 and j¹ is a value from 0 to 500, preferably 0 to 300.

In a particularly preferred embodiment, PDMS1 (R¹⁵=CH₃, R¹⁶=(CH₂)₃, R¹⁷=CH₃, h¹=2, j¹=13) is used as monomer (E).

Single compounds corresponding to general Formula (VIII) as well as mixtures of these compounds can be employed.

Average molecular weight of such monomers (also called “silicone macromers”), as measured by GPC (gel permeation chromatography), is preferably 100 to 40,000 g/mol, particularly preferably 200 to 20,000 g/mol.

### Monomers (F)—

Inventively usable polyfunctional monomer (F) is a monomer containing polymerizable functional groups, with monomers possessing two or three functional groups preferably used. These molecules can contribute to formation of bridges and branches within the copolymer and are thereby especially suitable for extending the duration of attachment and thereby also the desired anti-adhesive effect.

In a preferred embodiment, the inventively usable polyfunctional monomer (F) comprises one or more hydrophilic groups.

General Formula (IX) illustrates a typical example of a bifunctional monomer, whereas general Formula (X) represents a typical example of a trifunctional monomer—

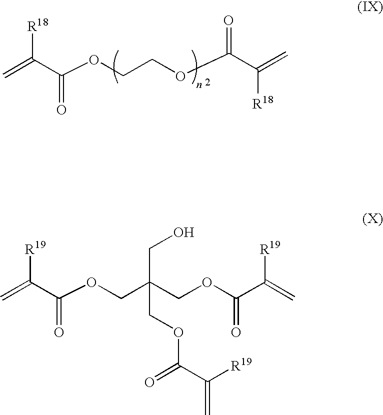

In general Formulae (IX) and (X), groups R¹⁸ and R¹⁹ are each independently hydrogen, methyl or ethyl. In Formula (IX) n² is a value from 1 to 20.

Monomers according to Formula (IX) are preferably employed as the polyfunctional monomer (F), wherein the R¹⁸ groups are each independently hydrogen or methyl, and n² is a value of 1 to 15. In a particularly preferred embodiment, the compound PEG400DA (R¹⁸=H, n=8) is employed.

Polyfunctional monomers can also be vinyl monomers having at least one alkoxysilane group as illustrated in general Formula (XI), or methylol derivatives as illustrated in general Formula (XII).

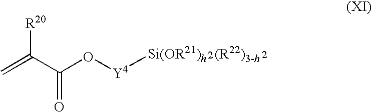

In general Formula (XI) R²⁰ is hydrogen or methyl, R²¹ and R²² are each independently aliphatic hydrocarbons containing preferably 1 to 6 carbon atoms, in particular methyl or ethyl, Y⁴ is an alkylene group containing 1 to 6 carbon atoms, in particular methylene or ethylene, and h² is 1, 2 or 3.

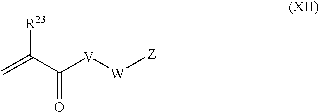

In general Formula (XII), R²³ is hydrogen, methyl or ethyl, V is O or NH, W is a hydrocarbon group containing 1 to 15 carbon atoms, in particular 1 to 6 carbon atoms, preferably methylene, ethylene, propylene or butylene, Z is OR²⁴, NHR²⁴, COOH, Br, epoxyethylene or NCO, and R²⁴ is hydrogen or a hydrocarbon group containing 1 to 6 carbon atoms.

The present invention furthermore concerns copolymers obtained by copolymerization of the following ethylenically unsaturated monomers:
65-95 wt. % of at least one anionic vinyl monomer (A), preferably AMPS;
0-5 wt. %, preferably 0-2 wt. %, of at least one vinyl monomer (B) containing a secondary or tertiary amino group or a quaternary ammonium group;
5 to 25 wt. % of at least one non-ionic hydrophilic vinyl monomer (C), preferably a PEG methyl ether (meth)acrylate, especially PEG methyl ether methacrylate 2080;
0-2 wt. % of at least one polyfunctional vinyl monomer (F);
0-2 wt. % of at least one hydrophobic vinyl monomer (D); and
0-2 wt. % of at least one vinyl monomer (E) comprising at least one silicone group;
   wherein the sum of monomers (A) and (C) is 80 wt. % or greater, preferably 90 wt. % or greater, and the sum of monomers (A), (B), (C), (D), (E) and (F) is 100 wt. %, wherein in a preferred embodiment, the sum of monomers (A) and (C) is at least 98 wt. %, preferably 100 wt. %.

The present invention furthermore concerns copolymers obtained by copolymerization of the following ethylenically unsaturated monomers:
5-30 wt. %, preferably 15-25 wt. %, of at least one anionic vinyl monomer (A), preferably AMPS;
0-5 wt. %, preferably 0-2 wt. %, of at least one vinyl monomer (B) containing a secondary or tertiary amino group or a quaternary ammonium group;
70 to 95 wt. %, preferably 75-85 wt. %, of at least one non-ionic hydrophilic vinyl monomer (C), preferably a PEG methyl ether (meth)acrylate, particularly preferably PEG methyl ether methacrylate 2080;
0 to 2 wt. % of at least one polyfunctional vinyl monomer (F);
0-2 wt. % of at least one hydrophobic vinyl monomer (D); and
0-2 wt. % of at least one vinyl monomer (E) comprising at least one silicone group;
   wherein the sum of monomers (A) and (C) is 80 wt. % or greater, preferably 90 wt. % or greater, and the sum of monomers (A), (B), (C), (D), (E) and (F) is 100 wt. %, wherein in a preferred embodiment, the sum of monomers (A) and (C) is at least 98 wt. %, preferably 100 wt. %.

Preferably, the present invention concerns a copolymer obtained by copolymerization of the following monomers:
5-30 wt. %, preferably 15-25 wt. %, above all 18-22 wt. %, of at least one anionic vinyl monomer (A), preferably AMPS;
70 to 95 wt. %, preferably 75-85 wt. %, above all 78-82 wt. %, of at least one non-ionic hydrophilic vinyl monomer (C), preferably a PEG methyl ether (meth)acrylate, preferably PEG methyl ether methacrylate 2080;
   wherein the sum of monomers (A) and (C) is preferably at least 99 wt. %, particularly preferably 100 wt. %.

The present invention concerns above all those copolymers explicitly cited in the experimental embodiments.

According to a preferred embodiment, the copolymers are employed in such final concentrations that they do not act as biocides or bacteriostatic agents. A particular advantage of this embodiment is that the risk of resistance development is low because the microorganisms that are present are neither killed off nor is their growth inhibited, the effect being purely biorepulsive. Concentrations for which no growth inhibition occurs, as well as the minimum inhibition concentration itself, can be easily determined by methods known to the person skilled in the art. It could be determined experimentally that many of the inventive copolymers showed no or only little bactericidal action, even when used in relatively high concentrations. Moreover, as far as is presently known, the majority of the inventive copolymers are harmless also from a toxicological viewpoint.

A further advantage of the invention is that some inventive copolymers, even compared to conventional biocides or bacteriostatic agents, are already effective in low final concentrations, so that only a low amount of substance needs to be used.

In a preferred embodiment, an inventive copolymer is used as an anti-fouling agent and/or in an anti-fouling agent.

According to a preferred embodiment, adhesion of microorganisms to filter media, adhesives, building materials and/or building auxiliaries is reduced.

In a further preferred embodiment, adhesion of microorganisms on surfaces that often come into contact with the human body is reduced. Here, in particular, are meant abiotic, industrial (or industrially manufactured) surfaces. In the scope of this particular embodiment, human or animal tissue is therefore understood not to be included.

According to a further preferred embodiment, adhesion of microorganisms on such surfaces as textiles, ceramics, metals, glass and/or plastics, is reduced. In particular they can concern washing, sanitary devices such as showers, wash basins or toilets, floor coverings, shoes, leather, articles of daily use, window panes, glasses, aquaria, dishes, work surfaces, prostheses, dental prostheses or kitchen equipment such as fridges or ovens. In this regard, the attachment and/or the formation of a biofilm is particularly preferably suppressed and/or reduced, especially on the previously mentioned hard surfaces, particularly preferably on ceramics, above all in the sanitary area.

In the applications mentioned, the inventive copolymers are preferably deposited onto the material or incorporated or inserted into the material.

Reduction in adhesion to textiles or plastic surfaces reduces the risk of a re-infection of the affected body region. Reduction in adhesion of microorganisms to ceramics, plastics or metals, particularly prosthetics or dentures, diminishes the risk of infection or re-infection, without polluting the skin, the mucous membranes or the waste water with biocidal or bacteriostatically or virostatically active substances. By the same token, catheters as well as other medical instruments manufactured from plastics or metals, and/or prosthetics, can be freed of adhesion by the use of inventive copolymers, for example, in rinse or cleaning agents.

Dentures, particularly sets of teeth, can be effectively cleaned from adhesion from microorganisms by use of the inventive copolymers in oral, dental and or denture care products, simply and without stressing the treated surface with strongly active biocides, potentially even proven toxic substances.

In a preferred inventive embodiment, adhesion of microorganisms is suppressed by attacking the molecular communication of the microorganisms, thereby inhibiting formation of a biofilm.

Accordingly, the present invention further provides a method for controlling processes based on microbial interaction, wherein
a) where necessary the interacting microorganisms are determined,
b) where necessary the appropriate compound or appropriate compounds are selected from the inventive copolymers, and
c) the selected compound or selected compounds are added in an amount sufficient for the desired control to the medium in which the microbial interaction takes place.

Accordingly, the invention provides use of an inventive copolymer for controlling processes based on microbial interaction, especially for controlling development and/or maturation of biofilms, particularly preferably of biofilms in which gram-negative bacteria are involved.

The “processes based on microbial interaction” are understood to mean, in addition to the development and/or maturation of biofilms, for example, also multicellular swarm behavior, the concerted development of antibiotic resistances, the concerted synthesis of antibiotics, the concerted synthesis of pigments, the concerted production of extracellular enzymes, in particular hydrolytic enzymes, or the concerted production of virulence factors.

The suppression of biofilm also indirectly protects, for example, ships' hulls against the growth of algae. The biofilm forms the basis for the settlement of larger organisms such as mussels and algae. This growth, due to its viscous drag, slows down the ship and thereby leads to an increased fuel consumption, as a result of which the deposits have to be periodically removed at great expense. For this reason the use of the inventive condensation polymers as and/or in so-called antifoulants is inventively particularly preferred.

Medically relevant biofilms are likewise a preferred aim of the present invention. In particular, cystic fibrosis, dental plaque as well as biofilms on contact lenses, implants and catheters should be cited.

Accordingly, in a preferred embodiment, use according to the invention is carried out to suppress biofilms in sterilization agents, disinfectants, impregnation agents or preservatives, washing or cleaning agents, or in coolants or cooling lubricants (technical application solutions) as well as in the field of water purification/water treatment, and the pharmaceutical, food, brewing, medical, colorant, wood, textile, cosmetic, leather, tobacco, hide, rope, paper, pulp, plastic, fuel, oil, rubber or machine industries.

In another preferred embodiment, the inventive use is for biofilm control for medical equipment, instruments and apparatuses, particularly for catheters and endoscopes.

### Microorganisms—

Microorganisms are understood to mean in particular bacteria, fungi, protozoa, viruses and microalgae. This includes bacterial endospores and exospores as well as spores that serve as reproduction structures in fungi. In a preferred embodiment, microorganisms are understood to mean bacteria and fungi. Particularly preferred fungi are here yeasts, molds, dermatophytes and keratinophilic fungi.

According to a particularly preferred embodiment, adhesion of bacteria is reduced by use of the inventive copolymers, particularly adhesion of gram-negative and gram-positive bacteria, principally adhesion of pathogenic bacteria chosen from Propionibacterium acnes, Staphylococcus aureus, Streptococcus of group A (beta-hemolytic S.), S. pyogenes, Corynebacterium spp. (particularly C. tenuis, C. diphtheriae, C. minutissimum), Micrococcus spp. (particularly M. sedentarius), Bacillus anthracis, Neisseria meningitidis, N. gonorrhoeae, Pseudomonas aeruginosa, P. pseudomallei, Borrelia burgdorferi, Treponema pallidum, Mycobacterium tuberculosis, Mycobacterium spp., Escherichia coli as well as Streptococcus spec. (particularly S. gordonii, S. mutans), Actinomyces spec. (particularly A. naeslundii), Salmonella spec., Actinobacteria (particularly Brachybacterium spec.), alpha-Proteobacteria (particularly Agrobacterium spec.), beta-Proteobacteria (particularly Nitrosomonas spec.), Aquabacterium spec., Hydrogenophaga, gamma-Proteobacteria, Stenotrophomonas spec., Xanthomonas spec. (campestris), Neisseria spec., Haemophilus spec. as well as all microorganisms that are described by Paster et al. (J. Bac. 183 (2001) 12, 3770-3783).

According to another preferred embodiment, use of the copolymers reduces adhesion of human pathogenic fungi. These include, for example, the human pathogenic species of fungi from the classes Ascomycota, Basidiomycota, Deuteromycota and Zygomycota, in particular, all species from the genera Aspergillus, Penicillium, Cladosporium and Mucor as well as Stachybotrys, Phoma, Alternaria, Aureobasidium, Ulocladium, Epicoccum, Stemphyllium, Paecilomyces, Trichoderma, Scopulariopsis, Wallemia, Botrytis, Verticillium and Chaetonium as well as the human pathogenic forms of Candida.

According to another preferred embodiment, adhesion of fungi of the species Rhodotorula spp., Cryptococcus spp., Exophilia spp., Hormoconis spp. is reduced.

According to the invention, adhesion of medically relevant forms of Candida is particularly preferably reduced, for example, C. albicans, C. boidinii, C. catenulata, C. ciferii, C. dubliniensis, C. glabrata, C. guilliermondii, C. haemulonii, C. kefyr, C. krusei, C. lipolytica, C. lusitaniae, C. norvegensis, C. parapsilosis, C. pulcherrima, C. rugosa, C. tropicalis, C. utilis, C. viswanathii. Particularly preferred are C. albicans, C. stellatoidea, C. tropicalis, C. glabrata and C. parapsilosis. The mycel form of Candida is considered as the human pathogenic form of the fungus. The reduction in adhesion of Candida to textiles or plastics, for example, reduces the risk of re-infection, without increasing the development of resistance.

The copolymers are particularly preferred for reducing adhesion of all species of the genera Aspergillus on surfaces, quite particularly preferred for species chosen from Aspergillus aculeatus, A. albus, A. alliaceus, A. asperescens, A. awamori, A. candidus, A. carbonarius, A. cameus, A. chevalieri, A. chevalieri var. intermedius, A. clavatus, A. ficuum, A. flavipes, A. flavus, A. foetidus, A. fumigatus, A. giganteus, A. humicola, A. intermedius, A. japonicus, A. nidulans, A. niger, A. niveus, A. ochraceus, A. oryzae, A. ostianus, A. parasiticus, A. parasiticus var. globosus, A. penicillioides, A. phoenicis, A. rugulosus, A. sclerotiorum, A. sojae var. gymnosardae, A. sydowi, A. tamarii, A. terreus, A. terricola, A. toxicarius, A. unguis, A. ustus, A. versicolor, A. vitricolae and A. wentii. Particularly preferably, adhesion of Aspergillus flavus and Apsergillus nidulans is reduced or essentially prevented.

According to another preferred embodiment, adhesion of keratinophilic fungi chosen from Trichophyton mentagrophytes, T. rubrum, T. asteroides, T. concentrium, T. equinum, T. meginii, T. gallinae, T. tonsurans, T. schoenleinii, T. terrestre, T. verrucosum, T. violaceum, Microsporum canis, Microsporum audounii, M. gypseum, Epidermophyton flossocum, Malassezia furfur, M. sympodialis, M. globosa and M. pachydermatis, is reduced.

According to another embodiment, the use of the copolymers reduces adhesion of algae, of human, animal and/or vegetal pathogenic viruses, as well as bacteriophages, particularly reduction of adhesion of green and blue algae on facades and building materials. The relevant members of the blue algae (cyanobacteria) are of the genera Anabaena, Anacystis (e.g., Anacystis Montana), Gloeocapsa, Lyngbia, Nostoc, Oscillatoria, (e.g., Oscillatoria lutea), Phormidium, Schiszothrix and Scytonema. Genera of the green algae (chlorophyta) include Chlorella, Choricystis, Chlamydomonas, Chlorococcum, Stichcoccus, particularly Stichcoccus bacillaris, Ulothrix and Trentepholia, particularly Trentepholia odorata.

Microorganisms in regard to biofilm formation that are particularly relevant and whose adhesion is particularly preferably reduced include Aeromonades, particularly Aeromonas hydrophila or Aeromonas salmonicida, Agrobacterium, particularly Agrobacterium tumefaciens, Aquabacterium, Bradyrhizobium japonicum, Burkholderia cepacia, Chromobacterium violaceum, Dermacocci, in particular Dermacoccus nishinomiyaensis, Enterobacter agglomerans, Erwinia carotovora, Erwinia chrysanthemi, Escherichia coli, Nitrosomona europaea, Obesumbacterium proteus, Pantoea stewartii, Pseudomonaden, particularly Pseudomonas aeruginosa, Pseudomonas aureofaciens, Pseudomonas fluorescens or Pseudomonas syringae, Ralstonia solanacearum, Rhizobium, particularly Rhizobium etli or Rhizobium leguminosarum, Rhodobacter sphaeroides, Salmonella enterica, Serratia, particularly Serratia liquefaciens, Vibrio anguillarum, Vibrio fischeri, Xanthomonas, particularly Xanthomonas campestris, Xenorhabdus nematophilus, Yersinia, particularly Yersinia enterolytica, Yersinia pestis, Yersinia pseudotuberculosis or Yersinia ruckeri.

Relevant biofilm builders in the marine environment which can contribute to causing so-called fouling on submersed surfaces, and whose adhesion and biofilm formation is likewise particularly preferably reduced, include Zooshikella gangwhensis, Pseudomonas fluorescens, Cythophaga sp. KT0803, Psychrobakter glacinola, Pseudoalteromonas carragenovora, Shewanella baltica and Bacillus subtilis.

### Washing and Cleaning Agents—

Use of the inventive copolymers is preferably carried out in washing and/or cleaning agents.

In addition to use of the inventive copolymers in washing and/or cleaning agents, the present invention also includes washing and/or cleaning agents comprising the previously described inventively preferred copolymers. The washing and/or cleaning agents are described in more detail below.

The washing and cleaning agents can comprise relatively low amounts of inventive copolymers without polluting the wastewater. As they are used in concentrated form and are diluted to the corresponding active concentrations in the wash liquor, the active substances in this case have to be used in a correspondingly higher concentration. Washing and cleaning agents are normally diluted with water in a ratio of 1:40 to 1:200.

According to the invention, the copolymer is preferably added to cleaning agents for cleaning hard surfaces such as floors, tiles, wall tiles, and plastics, as well as other hard surfaces in the household, in toilets, in public sanitary facilities, in swimming baths, saunas, sports facilities or in medical or massage practices.

In the broadest sense of the context of the invention, washing and cleaning agents are understood to mean surfactant-containing preparations in solid form (particles, powder etc.), semi-solid form (pastes etc.), liquid form (solutions, emulsions, suspensions, gels etc.) and gaseous-like form (aerosols etc.) that in regard to an advantageous effect in the application can also comprise any type of surfactant, usually in addition to further components that are usual for each of the end uses. Examples of such surfactant-containing preparations are surfactant-containing detergent preparations, surfactant-containing cleansing agents for hard surfaces, or surfactant-containing freshening preparations, each of which can be solid or liquid, however, they can also be in a form that includes solid and liquid components or partial amounts of the components alongside one another.

Washing and cleaning agents typically comprise ingredients such as anionic, non-ionic, cationic and amphoteric surfactants, inorganic and organic builders, special polymers (for example those with cobuilder properties), foam inhibitors, colorants and optional fragrances (perfumes), pH adjustors, thickeners, polyethylene glycols, bleaching agents (such as for example peroxy bleaching-agents and chlorine bleaching agents), bleach activators, bleach stabilizers, bleach catalysts, enzymes, in particular proteases, amylases or cellulases, enzyme stabilizers, color transfer inhibitors and anti-graying inhibitors, without the ingredients being limited to these groups of substances. Frequently, important ingredients of these preparations are also detergent auxiliaries, which are understood to include in a non-limiting sense as examples, optical brighteners, UV-stabilizers, soil repellents, i.e. especially polymers that counteract redeposition of dirt on the fibers. For the case where at least part of the preparations are present as molded bodies, binding auxiliaries and disintegration auxiliaries can also be comprised. In regard to the individual substance groups, reference is particularly made to the published contents of the application DE102007058342.9.

The inventive copolymers are present in the inventive agents, especially in inventive washing and/or cleaning agents, preferably in an amount of 0.01 to 10 wt. %, particularly preferably in an amount of 0.05 to 2 wt. %, especially in an amount of 0.1 to 1 wt. % based on weight of the agent.

Inventive washing and/or cleaning agents can exhibit an acidic, neutral or basic pH. In a preferred inventive embodiment, the inventive washing and/or cleaning agents have a pH of 0 to 14, particularly preferably from 0 to 7, especially from 1 to 4.

In an inventively particularly preferred embodiment, an inventive washing and/or cleaning agent, in particular a cleaner for hard surfaces, comprises:
0.1 to 10 wt. %, preferably 0.5 to 5 wt. %, in particular 1 to 3 wt. %, of at least one inventive copolymer,
0.01 to 10 wt. %, preferably 0.1 to 5 wt. %, of at least one polyethylene glycol with an average mean molecular weight of 200 to 600 000 g/mol, preferably 10 000 to 200 000 g/mol,
0.01 to 10 wt. %, preferably 0.1 to 3 wt. %, of at least one surfactant, preferably at least one anionic or non-ionic surfactant,
0 to 10 wt. %, preferably 0.01 to 10 wt. % of at least one thickener,
0.01 to 80 wt. %, preferably 0.1 to 5 wt. % of at least one organic solvent,
0.01 to 10 wt. %, preferably 0.1 to 3 wt. % of at least one complexant and/or builder,
0.01 to 10 wt. %, preferably 0.2 to 5 wt. %, of at least one inorganic or organic acid,
0.001 to 10 wt. %, preferably 0.01 to 1 wt. %, of at least one fragrance, and
0.001 to 10 wt. %, preferably 0.02 to 1 wt. %, of at least one colorant.

In another particularly preferred embodiment, an inventive washing and/or cleaning agent, particularly a cleaner for hard surfaces, is one wherein—
the surfactant is chosen from alkyl polyglycosides, especially C₈₋₁₀ alkyl-1,5-glucoside, sodium lauryl ether sulfate and sodium lauryl sulfate;
the organic solvent is chosen from alkanols, especially ethanol, propylene glycol, glycol ethers and benzene;
the builders are chosen from sodium citrate, sodium carbonate and phosphates;
the thickeners are chosen from polysaccharides, substituted cellulose, especially hydroxypropyl methyl cellulose, poly(meth)acrylates, guar gum and Xanthane derivatives such as Xanthane gum;
and the acid is chosen from citric acid, formic acid, lactic acid and amidosulfonic acid.

In another particularly preferred embodiment, an inventive washing and/or cleaning agent, in particular a cleaner for hard surfaces, has a pH of 0 to 10, preferably from 1 to 4.

### Pharmaceutical and Cosmetic Compositions—

Another subject matter of the present invention is the use of inventive copolymers in pharmaceutical and/or cosmetic compositions as well as the use of inventive copolymers for manufacturing cosmetic or pharmaceutical compositions, especially for treating bacterial or fungal infections.

The pharmaceutical compositions can be employed for both the treatment and also the prevention of illnesses.

For the manufacture of pharmaceutical preparations, active substances, optionally in combination with other active principals, can be incorporated with one or a plurality of inert, conventional carriers and/or diluents, e.g. with gelatin, gum arabic, corn starch, milk sugar, raw sugar, sorbitol, microcrystalline cellulose, magnesium stearate, polyvinyl pyrrolidone, citric acid, tartaric acid, water, benzyl alcohol, polyalkylene glycol, water/ethanol, water/glycerin, water/sorbitol, water/polyethylene glycol, propylene glycol, titanium dioxide, a cellulose derivative such as carboxymethyl cellulose or fat-containing substances such as hydrogenated fat, talcum or vegetal oils or their appropriate mixtures, in usual galenical preparations such as tablets, dragees, capsules, powders, suspensions, drops, ampoules, juices or suppositories. Optionally, preservatives, stabilizers, wetting agents, emulsifiers or salts for modifying the osmotic pressure or buffers can be comprised. Interfacially active auxiliaries such as salts of gallic acid or animal or vegetal phospholipids, mixtures thereof as well as liposomes or their constituents can also be used as the carrier.

The inventive pharmaceutical and cosmetic preparations can also comprise, in addition to the inventive active substances, active substances that prevent adhesion of microorganisms. Moreover, the use of the inventive active substances can also optionally be realized in combination with antimicrobials, particularly antibacterials, antimycotics and/or antiseptics and/or in combination with astringent substances, wherein the antimicrobials are then preferably employed in low concentrations.

In a particularly preferred embodiment according to the invention, the pharmaceutical or cosmetic preparations include those for topical application on skin and their adnexa and/or for application on the mucous membrane, particularly in the oral and genital region, or for intertriginous application. Such preparations are designated as “skin treatment agents”.

The cosmetic or pharmaceutical preparation, and particularly the skin treatment agent, can be in the form of a lotion, a cream, an emulsion, a balm, a paste, an oil, a wax/fat compound, a gel, a powder, a spray or aerosol, a solution, particularly aqueous or alcoholic solution, or tincture, a moist dressing, an occlusal dressing, a plaster, a stick preparation, a hair treatment, hair washing or hair care product, particularly a hair shampoo, a hair lotion, a hair cure or a hair water, a body care agent, a bubble bath, a shower bath or a foot bath.

The physiological carrier of the skin treatment agents advantageously includes one or any combination of a plurality of auxiliaries or additives, as are normally used in such preparations, such as fats, oils, greasing materials, waxes, silicones, emulsifiers, dispersants, pearlizers, alcohols, polyols, consistency agents, stabilizers, thickeners, film formers, swelling agents, hydrotropes or moisturizers and/or humectants, polymers, surfactants, plasticizers, defoamers, alkalisers or acidifiers, water softeners, adsorbents, light stabilizers, electrolytes, sequestering agents, solubilizers, organic solvents, preservatives, germicides, particularly fungicides or bactericides, antioxidants, biogenic active substances, vitamins, protein hydrolyzates, mono-, oligo- and polysaccharides, enzyme inhibitors, particularly MMP1-inhibiting substances, deodorants or odor absorbers, antiperspirants, antidandruff agents, insect repellents, self-tanning lotions, α-hydroxy- and α-ketocarboxylic acids, fragrances, colorants and/or pigments.

The inventive skin treatment agents are advantageously present for topical administration in the form of a liquid or solid oil-in-water emulsion, water-in-oil emulsion, multiple emulsion, micro-emulsion, PIT-emulsion or Pickering emulsion, in the form of a hydrogel, an alcoholic gel, a lipogel, in the form of a mono or multiphase solution, a foam, a balm, a plaster, a suspension, a powder or a mixture with at least one polymer that is a suitable medicinal adhesive. The inventive skin treatment agents can also be presented in an anhydrous state, such as in oil or a balsam. For this, the carrier can be vegetal or animal oil, a mineral oil, synthetic oil or a mixture of such oils.

In a further particularly preferred embodiment according to the invention, the cosmetic and/or pharmaceutical preparations concern those for oral application, wherein the target area of the application is the mouth. In a preferred embodiment here, one of the previously described skin treatment agents is used, wherein the composition is so chosen that the preparation concerns a mouth cream, a balm, a tincture or a suspension. The term, “pharmaceutical preparation for oral application” also includes, in addition to mouth and teeth care agents, prosthesis cleansing agents, particularly cleansing tablets for dentures.

The inventive oral, dental and/or dental prostheses care compositions can exist, for example, as mouth water, gels, liquid toothpaste, viscous toothpaste, denture cleaners or adhesive creams for prostheses. For this, the inventively used materials must be proposed in a suitable carrier.

The inventive toothpastes and tooth gels can comprise, in addition to the inventive active substances, particularly surfactants, cleaning compounds, aromas, sweeteners as well as additional active substances known to the person skilled in the art. Water and binders advantageously serve as the carriers. Furthermore, humectants, preservatives, consistency agents and/or color pigments, for example, can also be comprised.

In regard to the cited additional active substances that can be comprised in the oral treatment agents, they can concern, for example, a fluorine compound, an active substance against plaque bacteria, an active substance against calculus, for remineralization, against sensitive teeth or for the protection of the gums. Moreover, the additional active substance can concern an additional active substance for fungal treatment, particularly treatment of candidosis.

Additional typical additives for oral, dental and/or dental prostheses care agents include—
pH adjustors and buffer substances such as sodium bicarbonate, sodium citrate, sodium benzoate, citric acid, phosphoric acid or acidic salts (e.g., NaH₂PO₄)
Wound healing and anti-inflammatory substances such as allantoin, urea, panthenol, azulene or chamomile extract
Further active materials against tartar such as organo phosphonates (e.g., hydroxyethane diphosphonate or azacycloheptane diphosphonate)
Preservatives such as salts of sorbic acid, sodium benzoate, chlorhexidine digluconate, p-hydroxybenzoic acid or its esters
Plaque-inhibitors such as hexachlorophene, chlorhexidine, hexetidine, triclosan, bromochlorophene, phenyl salicylate

### EXAMPLES

### Example 1

### Synthesis of Inventive Copolymers

PEG-MA 2080: AMPS=80:20 (compound 9007-009) (parts by wt. %)

2-Acrylamido-2-methylpropanesulfonic acid (2.00 g) and polyethylene glycol methacrylate 2080 (16 g, 50% soln. in water) were weighed out into a 250 ml flask and dissolved in 62 g deionized water. The reaction mixture was degassed and the following reaction was carried out under nitrogen. The contents of the flask were then heated to 65° C. A solution of 2,2′-azobis(2-amidinopropane) dihydrochloride (V50) (0.2 g) in 0.8 g water was then added. The resulting mixture was stirred for one hour at 75° C. and then for a further hour at 80° C. A viscous polymer solution was obtained. The obtained reaction product can then be subjected to dialysis in order to remove residual monomer. Alternatively, a post-initiation step can be carried out during the reaction.

In cases where more hydrophobic monomers are employed, the use of surfactants and dispersion agents can be helpful. The pH of the reaction can also be adjusted before the polymerization or after the reaction and before carrying out application tests. Other water-soluble initiators that are thermo labile can also be used; alternatively, redox pairs or photoinitiators can also be used.

The following additional polymers were obtained in a similar manner (each fraction in wt. %): mixtures of

The monomers used are illustrated below—

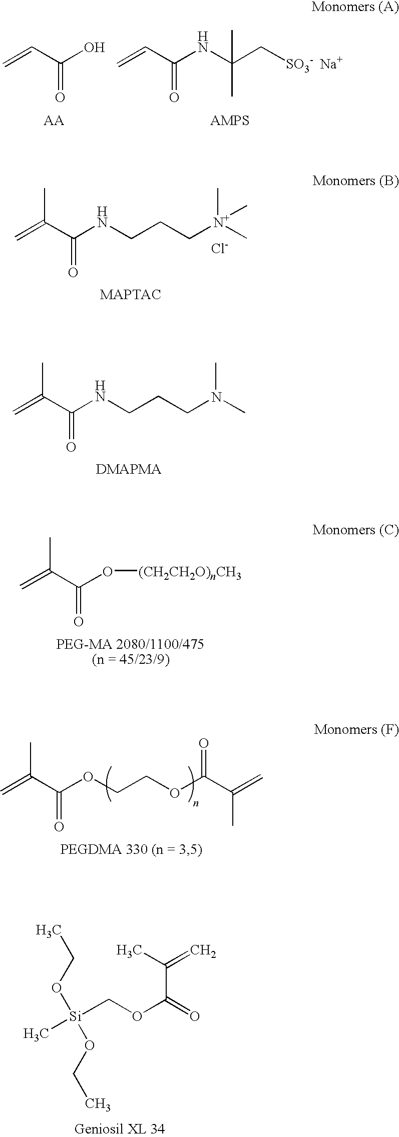

### Example 2

### Polymer Screening in the Adhesion Experiment

In order to compare the biorepulsive power of polymer films on hard surfaces relevant in the household (e.g., ceramics, plastic, stainless steel and glass), the polymers were initially tested in a screening approach. For this, adhesion tests for microorganisms were carried out (here: Staphylococcus aureus DSM799 and Pseudomonas aeruginosa DSM939). Specimens (glass, plastic, ceramic, stainless steel) sized 18×18 to 20×20 mm were first disinfected with 70% conc. methanol for 10 minutes and then washed with sterile and distilled water and dried. The thus-prepared specimens were coated with a germ suspension that additionally comprised an appropriate polymer concentration and incubated for 1 hour. The germ suspension was then aspirated off and the specimen washed two times. The specimens were then transferred in sterile test plates, coated with nutrient agar for S. aureus and then incubated at 30° C. for 48 hours. For P. aeruginosa the specimens were shaken in buffer solution, subsequently coated with nutrient agar plus 10% TZC and then incubated at 30° C. for 24 hours. The shaking liquid was filtered over a membrane and the filter incubated on Caso agar at 30° C. for 24 hours. The degree of germ growth, which can be attributed to the colonization of the specimens with germs, is listed relative to a culture without polymer but with the corresponding solvent fraction in %. In this regard, the germ loading of the control specimen is set at 100%. Table 1 shows the best acting polymers in the screening adhesion test, wherein an effective activity is always defined as a reduction in germs of at least 50% in comparison with the control. The best polymers have a broad biorepulsive action against both test germs with as many as possible surfaces and already have an optimal action at concentrations below 1%.

All polymers of Example 1 showed a good effect in this test, wherein very good results were obtained in regard to the tested microorganisms on the following surfaces (G, T, K and S stand for glass (G), plastic (T), ceramic (K) and stainless steel (S)):
9007-012: P. aeruginosa (G, T, K, S); S. aureus (G, T, K, S)
8406-108: P. aeruginosa (G, T, K, S); S. aureus (G, T)
8389-115: P. aeruginosa (G, T, K, S); S. aureus (G, K)
9007-001: P. aeruginosa (G, T, K, S); S. aureus (T)
8389-094: P. aeruginosa (G, T, K, S); S. aureus (T)
8389-036: P. aeruginosa (G, T, K, S)
8389-181: P. aeruginosa (G, T, K, S)
8844-046: P. aeruginosa (G, T)
8844-048: P. aeruginosa (G, T)
8844-004: P. aeruginosa (T, K)
9007-009: P. aeruginosa (T, K)
8406-102: P. aeruginosa (T, K)
   All polymers were employed in an amount of 1 wt. %.

### Example 3

### Polymer-Coated Surfaces in the Adhesion Experiment

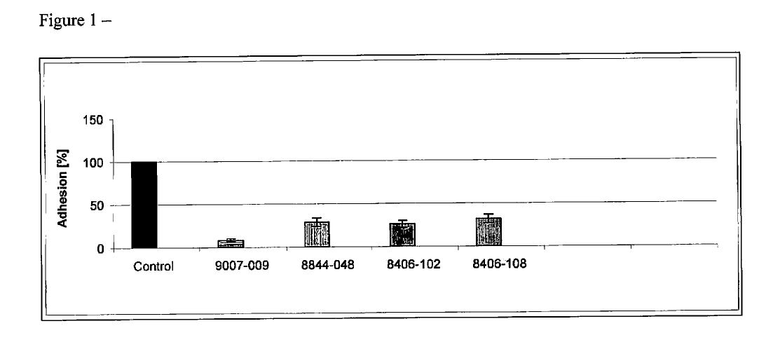

In order to eliminate interactions between the dissolved polymers and the test germs in the test cultures, the action of selected polymers was tested directly on the surface. The polymers were immobilized as follows: 1% conc. polymer solution in ethanol, 40 μl of this polymer solution were coated onto surfaces (plastic and ceramic) and dried at room temperature for 24 hours (control: only ethanol). The thus-prepared specimens were coated with a germ suspension of S. aureus and incubated for 1 hour. The degree of germ growth which can be attributed to the colonization of the specimens with germs is listed relative to a specimen coated with the comparative composition in %. In this regard, the germ loading of the specimen coated with the control composition is set at 100%. It was observed that most of the immobilized polymers demonstrated the same action as in the dissolved form. In FIG. 1 the results for the polymers 9007-009, 8844-048, 8406-102 and 8406-108 are illustrated as examples. It is observed that the polymers cause a significant reduction in adhesion.

### Example 4

### Polymer-Coated WC (Water Closet) Ceramic in the Laboratory Test Under Approximately Real-Life Conditions

The polymers that demonstrated a significant germ reduction in the simplified test method (especially on ceramic) were subsequently tested in a test system under approximately real-life conditions that simulate the function of a toilet. In order to compare the biorepulsive power of polymer films on WC ceramics, it was necessary to decide on uniform test conditions. For this purpose a test method including germ loading was developed, which corresponds to actual conditions in the toilet. As in the actual toilet, the flush over the test ceramic was made from a water tank by opening a valve. The curvature of the toilet bowl was reproduced by means of an inclined plane with an angle of 45° and flat test tiles from Villeroy & Boch (15×15 cm²). The sprinkler unit served to wet the test tiles as homogeneously as possible with 150 ml sterile service water per second. In general, 900 ml water was used per flush. The test tile was treated with ethanol before the experiment, then the test polymer was added (2 ml undiluted polymer was rubbed with cellulose pulp onto the tile) and then dried horizontally at room temperature for 60 minutes to form the polymer film. The inclined tile was then homogeneously loaded with an S. aureus suspension (10⁴ germs in 100 ml table salt solution) and incubated at room temperature for 10 minutes. The anti-adhesive action was determined by wetting the tile with sterile service water from the sprinkler unit. In order to record the residual germ count on the tile, a central RODAC copy was carried out on the tile after the flush. The flush step including each renewed germ soiling and the associated RODAC analyses were repeated for each polymer being tested so as to examine the biorepulsive action also after multiple flushes (i.e. elution). The RODAC plates were incubated at 37° C. overnight and then quantitatively evaluated.

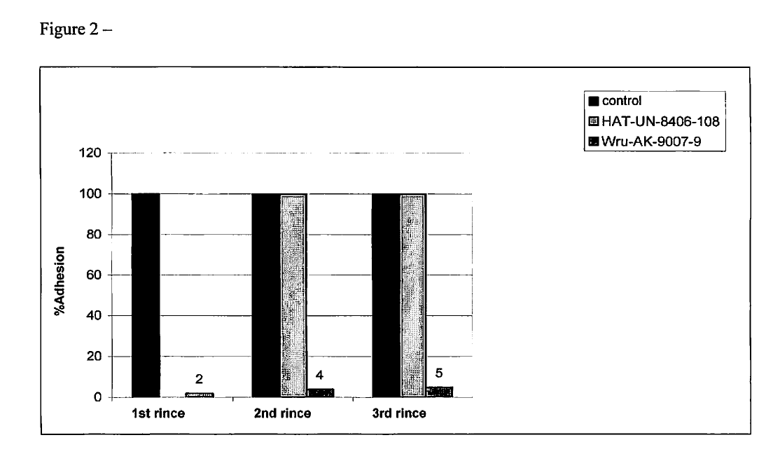

It was surprisingly found after the flush steps that a significant reduction of adhesion of microorganisms to the ceramic could be achieved by certain polymers, even after multiple flush steps. In spite of the elution of the polymer layer on the tile surface, the binary AMPS/PEG polymer 9007-009 in particular showed a more than 90% reduction of the germ adhesion even after 10 flush cycles (FIG. 2, per flush step each of the right bars). In contrast, the polymer 8406-108 (per flush step each of the middle bars) showed an even better action than 9007-009 for the first flush cycle, but this action was completely lost already in the second flush cycle, demonstrating that the polymer is completely washed off in the first flush cycle.

### Example 5

### Laboratory Test Under Approximately Real-Life Conditions in the WC Reactor on Ceramic Tiles

In parallel the ceramic tiles were examined in an almost automatically running WC reactor under approximately real-life conditions which was designed to simulate the function of a toilet. This system allows adhesion and biofilm formation to be investigated in a test system on a plurality of different surfaces over a short as well as a longer period (here: total running time of three days). In addition and in contrast to the microtiter plate system, it is a dynamic system because continuous fresh medium (TBY/DGHM water 1:50) is run over the tiles. In addition the surfaces become dried in phases and are then coated again with liquid. This change very strongly mimics the activities in a toilet, where the ceramic surfaces are intermittently wetted and can dry off again. The biofilms produced in the reactor correspond in regard to strength and homogeneity to those from microtiter plates.

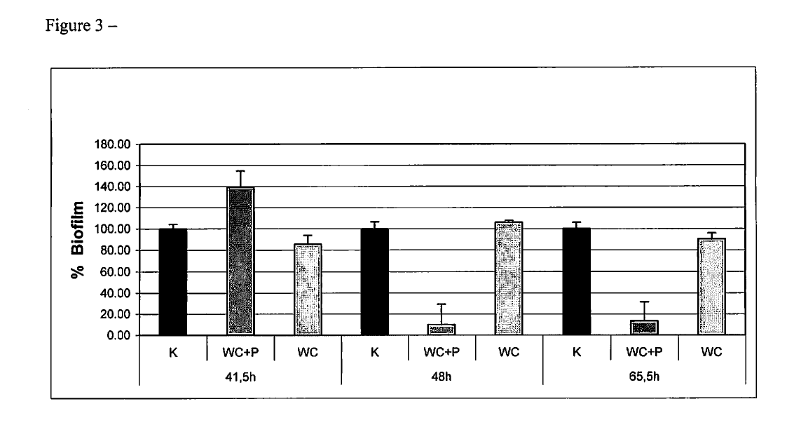

The reactor was first filled with 680 ml medium and impregnated with a germ mixture consisting of Dermacoccus nishinomiyaensis DSMZ 20448, Bradyrhizobium japonicum DSMZ 1982 and Xanthomonas campestris DSMZ 1526, which forms a stable biofilm in aqueous surroundings. The incubation took place overnight, in order for the bacterial flora to be able to establish itself in the system. As in the actual toilet, the flush over the test ceramic was made from a reservoir by opening a magnetic valve that was again controlled by a time switch. The curvature of the toilet bowl was reproduced by clamping the tiles by means of an adaptor in the interior of the reactor. In general, ca. 600 ml water was used per flush. 15 flushes were made on each of the first and second days after the incubation, wherein the single flush cycle lasted for 20 minutes. The first tiles were removed on the morning of the first day, after which still no or few flushes occurred. The second removal was made in the afternoon after the flushes; over night the reactor was filled with medium without any following flushes. Before being clamped in the reactor, the horizontally placed tiles were sprayed with a commercial WC cleaner comprising a 10% conc. polymer solution, 6 spray shots each being used per tile. After having been removed from the reactor the ceramic tiles were dried at room temperature and then each dyed with 6 ml 0.01% conc. safraninO solution for 15 minutes. The dye solution is then aspirated away, the non-bonded dye is removed from the tiles with bidistilled water and the dyed tiles are dried. The dyed and dried surfaces of the tiles were scanned and evaluated using Corel Draw Paint 9. The results for the polymer 9007-009 are presented in FIG. 3 in comparison with an untreated surface and in comparison with a surface treated solely with WC cleaner. It is noted that the WC cleaner that contains polymer effects a significant reduction of the biofilm, not only against the control tile that was neither treated with polymer nor with WC cleaner, but also against the tile that was solely treated with WC cleaner. After 65 hours an almost 70% reduction in biofilm was noted in comparison with non-coated controls.

### Formulation Examples

### Formulation 1

Aqueous solution comprising 1 wt. % of an inventive copolymer (preferably consisting of 20 wt. % AMPS and 80 wt. % PEG-MA 2080), 3 wt. % citric acid, 0.5 wt. % formic acid, 0.5 wt. % Kelzan ASX-T (xanthane gum from CP Kelco), 3 wt. % ethanol, 1 wt. % Texapon NSO (lauryl ether sulfate, sodium salt from Cognis France SA.), 0.002 wt. % patent blue (dye) and 0.02 wt. % of a fragrance.

### Formulation 2

Aqueous solution comprising 2 wt. % of an inventive copolymer (preferably consisting of 20 wt. % AMPS and 80 wt. % PEG-MA 2080), 3 wt. % citric acid, 2 wt. % ethanol, 1 wt. % Texapon NSO (lauryl ether sulfate, sodium salt from Cognis France SA.), 0.002 wt. % patent blue (dye) and 0.02 wt. % of a fragrance.

### Formulation 3

Aqueous solution comprising 1 wt. % of an inventive copolymer (preferably consisting of 20 wt. % AMPS and 80 wt. % PEG-MA 2080), 1 wt. % monoethanolamine (MEA), 2 wt. % ethanol, 0.6 wt. % Texapon LS (fatty alcohol sulfate, sodium salt from Cognis Germany GmbH), 0.002 wt. % patent blue (dye) and 0.02 wt. % of a fragrance.

### FIGURES

In FIG. 1 the results of the adhesion test described in Example 3 are presented. The cited polymers were coated onto plastic surfaces and the adhesion of Staphylococcus aureus was then investigated in comparison with an untreated plastic surface. The quantity of adhering bacteria was shown in percent, the adhesion on the untreated surface being set to 100%.

FIG. 2 provides results of the adhesion test under approximately real-life conditions in the laboratory test described in Example 4 with Staphylococcus aureus with polymer-coated ceramic tiles for the polymers 9007-009 (right bars) and 8406-108 (left bars) in comparison with a reference that was not treated with polymer (left bars, set to 100%). Whereas with the polymer 9007-009 could still effect a significant reduction in adhesion of Staphylococcus aureus even after three flush cycles, the polymer 8406-108 only effected an almost complete reduction of the adhesion of Staphylococcus aureus during the first flush cycle; this is explainable by the fact that the polymer 8406-108 is already washed off in the first flush cycle, whereas the polymer 9007-009 adheres semi-permanently to the surface.

FIG. 3 provides results of the adhesion test under approximately real-life conditions in the laboratory test described in Example 5 in the WC reactor on ceramic surfaces using the polymer 9007-009. The results after 41.5, 48 and 65.5 hours incubation are presented. The quantity of the adhering cells on the tiles treated with neither WC cleaner nor with polymer was set to 100% in each case. It is noted that after 48 and 65.5 hours incubation the quantity of the adhering cells on the tiles treated with neither WC cleaner nor with polymer is significantly reduced, both in comparison with the tiles that were neither treated with WC cleaner nor with polymer, as well as with the tiles that were only treated with commercial WC cleaner.

We claim:

1. Method of reducing adhesion of microorganisms and reducing development of biofilms on a surface comprising applying to the surface a copolymer of ethylenically unsaturated monomers, wherein the monomers used in forming the copolymerized ethylenically unsaturated copolymers are chosen from:
5-95 wt. % of at least one anionic vinyl monomer (A);
0-50 wt. % of at least one vinyl monomer (B) containing a secondary or tertiary amino group or a quaternary ammonium group;
5-95 wt. % of at least one non-ionic hydrophilic vinyl monomer (C); and/or
0-15 wt. % of at least one polyfunctional vinyl monomer (F); as well as
0-30 wt. % of at least one hydrophobic vinyl monomer (D); and
0-20 wt. % of at least one vinyl monomer (E) comprising silicone groups;
wherein the sum of monomers (A), (B), (C), (D), (E) and (F) is 100 wt. %.

2. Method according to claim 1, wherein the copolymerized ethylenically unsaturated monomers are chosen from:
65-95 wt. % of at least one anionic vinyl monomer (A);
0-5 wt. % of at least one vinyl monomer (B) containing a secondary or tertiary amino group or a quaternary ammonium group;
5-25 wt. % of at least one non-ionic hydrophilic vinyl monomer (C);
0-2 wt. % of at least one polyfunctional vinyl monomer (F);
0-2 wt. % of at least one hydrophobic vinyl monomer (D); and
0-2 wt. % of at least one vinyl monomer (E) comprising silicone groups;
wherein the sum of the monomers (A), (B), (C), (D), (E) and (F) is 100 wt. %.

3. Method according to claim 1, wherein the copolymerized ethylenically unsaturated monomers are chosen from:
5-30 wt. % of at least one anionic vinyl monomer (A);
0-5 wt. % of at least one vinyl monomer (B) containing a secondary or tertiary amino group or a quaternary ammonium group;
70-95 wt. % of at least one non-ionic hydrophilic vinyl monomer (C);
0-2 wt. % of at least one polyfunctional vinyl monomer (F);
0-2 wt. % of at least one hydrophobic vinyl monomer (D); and
0-2 wt. % of at least one vinyl monomer (E) comprising silicone groups;
wherein the sum of the monomers (A), (B), (C), (D), (E) and (F) is 100 wt. %.

4. Method according to claim 2, wherein the sum of monomers (A) and (C) is at least 98 wt. %.

5. Method according to claim 1, wherein the average molecular weight of the copolymer is from 10,000 to 1,000,000 g/mol.

6. Method according to claim 1 wherein
anionic vinyl monomer (A) is chosen from acrylic acid (AA); methacrylic acid (MAA); monomer according to general Formula (I) 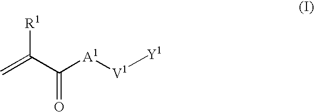
wherein R¹ is for hydrogen, methyl or ethyl, Y¹ is sulfato or sulfonato, A¹ is O or NH, and V¹ is a linear or branched, saturated or unsaturated hydrocarbon group containing 1 to 15 carbon atoms; monomerS according to Formula (II) 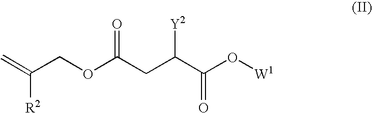
wherein, R² is hydrogen, methyl or ethyl, Y² is sulfato or sulfonato, W¹ is a linear, branched or alicyclic, saturated or unsaturated hydrocarbon group containing 1 to 20 carbon atoms; or the salts thereof;
vinyl monomer containing secondary or tertiary amino groups or quaternary ammonium groups (B) is chosen from monomers according to general Formula (III) 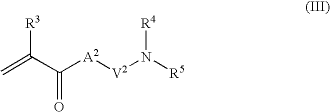
wherein R³ is hydrogen, methyl or ethyl, A² is O or NH, and V² is a linear or branched, saturated or unsaturated hydrocarbon group containing 1 to 15 carbon atoms, R⁴ is hydrogen, methyl, ethyl, propyl or butyl and R⁵ independently is methyl, ethyl, propyl or butyl; monomers according to general Formula (IV) 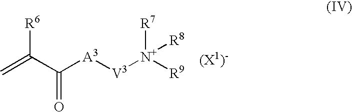
wherein R⁶ stands for hydrogen, methyl or ethyl, A³ for O or NH, V³ for a linear or branched, saturated or unsaturated hydrocarbon group containing 1 to 15 carbon atoms, X¹ represents a counter ion, R⁷, R⁸ and R⁹ independently of one another stand for methyl or ethyl; and monomers according to general Formula (V) 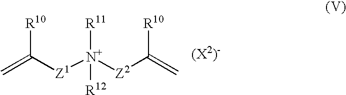
wherein each R¹⁰ is independently hydrogen, methyl or ethyl, R¹¹ and R¹² are each independently methyl or ethyl, X² is a counter ion, and Z¹ and Z² are each independently methylene, ethylene or linear propylene;
hydrophilic vinyl monomer (C) is a monomer according to general Formula (VI) 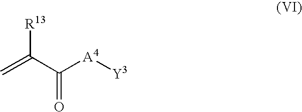
wherein R¹³ is hydrogen, methyl or ethyl, A⁴ is O or NH, Y³ is [(CH₂)ₘ₁O]ₙ₁B¹, wherein B¹ is hydrogen, methyl or ethyl, m¹ is a value from 2 to 4, and n¹ is a value from 1 to 60, and wherein for each [(CH₂)ₘ₁O] group the value for m¹ can be the same or different;
hydrophobic vinyl monomer (D) is a monomer according to general Formula (VII) 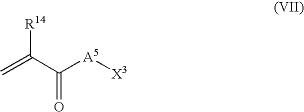
wherein R¹⁴ is hydrogen, methyl or ethyl, A⁵ is O or NH, and X³ is a linear or branched, saturated or unsaturated hydrocarbon group containing 1 to 15 carbon atoms;
vinyl monomers with a silicone group (E) are monomers according to general Formula (VIII) 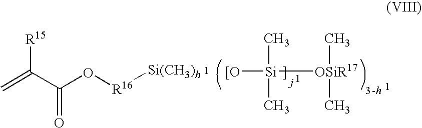
wherein R¹⁵ is hydrogen, methyl or ethyl, R¹⁶ is a linear or branched, saturated hydrocarbon group containing 1 to 6 carbon atoms, wherein one or more CH₂ groups in the hydrocarbon group can be optionally substituted with O, R¹⁷ is a linear or branched, saturated or unsaturated hydrocarbon group, wherein the hydrocarbon group can optionally be mono- or polysubstituted with fluorine, and wherein h¹ is 1 or 2 and j¹ is a value of 0 to 500;
polyfunctional vinyl monomer (F) is chosen from monomers according to general Formulae (IX), (X), (XI) and (XII) 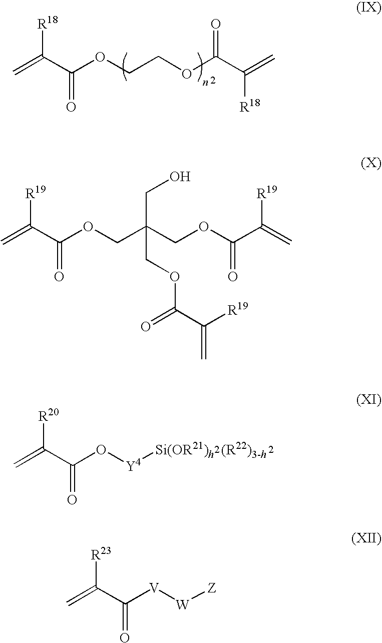
wherein in general Formulas (IX) and (X) R¹⁸ and R¹⁹ are each independently hydrogen, methyl or ethyl, and n² is a number from 1 to 20;
wherein in general Formula (XI) R²⁰ is hydrogen or methyl, R²¹ and R²² are each independently aliphatic hydrocarbon groups containing 1 to 6 carbon atoms, Y⁴ is an aliphatic hydrocarbon group containing 1 to 6 carbon atoms and h² is 1, 2 or 3; and
wherein in general Formula (XII) R²³ is hydrogen, methyl or ethyl, V is O or NH, W is a hydrocarbon group containing 1 to 15 carbon atoms, Z is OR²⁴, NHR²⁴, COOH, Br, epoxyethylene or NCO, and R²⁴ is hydrogen or a hydrocarbon group containing 1 to 6 carbon atoms.

7. Method according to claim 1, wherein the monomers used in forming the copolymerized ethylenically unsaturated copolymers are selected from the group consisting of anionic vinyl monomer (A) and hydrophilic vinyl monomer (C).

8. Copolymers obtained by copolymerization of ethylenically unsaturated monomers chosen from:
65-95 wt. % of at least one anionic vinyl monomer (A);
0-5 wt. %, of at least one vinyl monomer (B) having a secondary or tertiary amino group or a quaternary ammonium group;
5 to 25 wt. % of at least one non-ionic hydrophilic vinyl monomer (C);
0 to 2 wt. % of at least one polyfunctional vinyl monomer (F);
0-2 wt. % of at least one hydrophobic vinyl monomer (D); and
0-2 wt. % of at least one vinyl monomer (E) comprising at least one silicone group;
wherein the sum of monomers (A) and (C) is 80 wt. % or greater, and the sum of monomers (A), (B), (C), (D), (E) and (F) is 100 wt. %.

9. Copolymers obtained by copolymerization of ethylenically unsaturated monomers chosen from:
5-30 wt. % of at least one anionic vinyl monomer (A);
0-5 wt. %, of at least one vinyl monomer (B) having a secondary or tertiary amino group or a quaternary ammonium group;
70 to 95 wt. % of at least one non-ionic hydrophilic vinyl monomer (C);
0 to 2 wt. % of at least one polyfunctional vinyl monomer (F);
0-2 wt. % of at least one hydrophobic vinyl monomer (D); and
0-2 wt. % of at least one vinyl monomer (E) having at least one silicone group;
wherein the sum of monomers (A) and (C) is 80 wt. % or greater, and the sum of monomers (A), (B), (C), (D), (E) and (F) is 100 wt. %.

10. Copolymers obtained by copolymerization of ethylenically unsaturated monomers according to claim 9, wherein the sum of monomers (A) and (C) is 99 wt. % or greater.

11. Washing and/or cleaning agent comprising at least one copolymer according to claim 9.

12. Washing and/or cleaning agent according to claim 11, wherein the at least one copolymer is present in an amount of 0.1 to 10 wt. %, the washing and/or cleaning agent further comprising:
0.01 to 10 wt. % of at least one polyethylene glycol having an average mean molecular weight of 200 to 600,000 g/mol,
0.01 to 10 wt. % of at least one surfactant,
0 to 10 wt. % of at least one thickener,
0.01 to 80 wt. % of at least one organic solvent,
0.01 to 10 wt. % of at least one complexant and/or builder,
0.01 to 10 wt. % of at least one inorganic or organic acid,
0.001 to 10 wt. % of at least one fragrance, and
0.001 to 10 wt. % of at least one colorant,
weight based on total weight of the washing and/or cleaning agent.

13. Cosmetic or pharmaceutical preparation comprising at least one copolymer according to claim 9.

14. Method according to claim 1 wherein the at least one anionic vinyl monomer (A) is at least 2-acrylamido-2-methylpropane sulfonic acid.

15. Method according to claim 1 wherein the at least one non-ionic hydrophilic vinyl monomer (C) is at least PEG methyl ether methacrylate 2080.

16. Method according to claim 1 wherein the at least one anionic vinyl monomer (A) is present in an amount of 15 to 25 wt. % and the at least one non-ionic hydrophilic vinyl monomer (C) is present in an amount of 75 to 85 wt. %, weight based on total weight of the copolymer.
